(19) United States
(12) Patent Application Publication
(10) Pub. No.: US 2025/0084017 A1
(43) Pub. Date: Mar. 13, 2025

(54) ETHANOL DEHYDRATION PROCESS

(71) Applicant: Scientific Design Company, Inc., Little Ferry, NJ (US)

(72) Inventor: Saurabh KATIYAR, Little Ferry, NJ (US)

(73) Assignee: Scientific Design Company, Inc., Little Ferry, NJ (US)

(21) Appl. No.: 18/439,226

(22) Filed: Feb. 12, 2024

Related U.S. Application Data
(60) Provisional application No. 63/537,629, filed on Sep. 11, 2023.
(60) Provisional application No. 63/601,507, filed on Nov. 21, 2023.

(51) Int. Cl. C07C 5/333

(52) U.S. Cl. CPC C07C 5/3332; C07C 2521/04

(57) ABSTRACT

A process for the dehydration of ethanol to ethylene with reduced steam consumption and the use of latent heat of condensation to generate steam.

### CROSS-REFERENCE TO RELATED APPLICATIONS

This application claims benefit of priority to U.S. Provisional Application Ser. No. 63/537,629 filed Sep. 11, 2023 and U.S. Provisional Application Ser. No. 63/601,507 filed Nov. 21, 2023, the entire contents of which each are incorporated herein by reference.

### FIELD OF THE INVENTION

The present invention relates to a method for the dehydration of ethanol to ethylene and the oxidation of ethylene into ethylene oxide.

### BACKGROUND OF THE INVENTION

Found in trace amounts amidst the vast cosmic dust clouds, the molecule of ethylene oxide has long been a part of our universe's natural composition. It wasn't until 1859, however, that this molecule was synthesized on Earth, a feat achieved by Charles-Adolphe Wurtz through the pioneering chlorohydrin process. The full industrial potential of this compound remained latent until the rise of the automotive industry which increased demand for ethylene glycol, an antifreeze derived from ethylene oxide. This necessity catalyzed further developments, leading to Theodore Lefort's 1931 discovery of a more economically viable production method—the direct catalytic oxidation of ethylene.

Since then, ethylene oxide production has soared so that today it is among the most produced chemicals, with worldwide production reaching 34 billion tons by 2020. (Most of this ethylene oxide is further processed into derivatives such as ethylene glycol.) Concurrent with the rise in production, research into ethylene oxide catalysis and processing has flourished. A particular area of interest lies in developing more efficient and sustainable production processes, such as in the production of ethylene oxide from ethanol. Ethanol can be produced from renewable resources, presenting a sustainable alternative to traditional petrochemical-derived ethylene. The conversion process involves the dehydration of ethanol to form ethylene, which is subsequently oxidized to ethylene oxide.

While the use of ethanol as an alternative source of ethylene addresses sustainability concerns, traditional petrochemical-sourced ethylene, ethanol-based processes for ethylene oxide production are energy-intensive. The dehydration of ethanol is itself an endothermic reaction, meaning it requires the input of heat to proceed. This heat is usually supplied in the form of steam and conducted at dilute weight ratios of 3:1, steam to ethanol. The dilute dehydration reaction has the advantage of near 100% conversion and selectivity but at such dilute ratios the need for steam input is considerable. Thus, despite the environmental advantages of using a renewable feedstock like ethanol, the energy requirements undermine its sustainability benefits.

Accordingly, there is a continuing need for an ethanol dehydration process that combines its sustainability benefits with operation at high conversion and selectivity, and while also reducing its energy requirements from outside the process.

### BRIEF SUMMARY OF THE INVENTION

The present invention relates a process for the dehydration of ethanol to ethylene comprising the steps of: (a) forming a reactor feed mixture by blending an ethanol vapor stream with a superheated steam stream; (b) supplying the reactor feed mixture to the ethanol dehydration reactor; (c) dehydrating the ethanol in the reactor feed mixture in ethanol dehydration reactor to form an ethylene product stream; (d) supplying the ethylene product stream to a steam generator at a pressure of about 0.2 to about 0.3 MPa and a temperature of about 125° C. to about 140° C.; (e) cooling the ethylene product stream to below a dew point of the ethylene product stream to release the heat of condensation; (f) generating a second process steam stream by the heat of condensation, the second process steam stream having a temperature of about 95° C. to about 120° C.; (g) passing from the steam generator the process steam stream at a pressure of about 0.125 MPa to about 0.2 MPa; and (h) compressing the process steam stream to a pressure of about 0.3 MPa to about 0.45 MPa.

### BRIEF DESCRIPTION OF THE SEVERAL VIEWS OF THE DRAWINGS

The foregoing summary, as well as the following detailed description of preferred embodiments of the invention, will be better understood when read in conjunction with the appended drawings. For the purpose of illustrating the invention, there is shown in the drawings embodiments which are presently preferred. It should be understood, however, that the invention is not limited to the precise arrangements and instrumentalities shown. In the drawings:

FIG. 1 is a schematic flow sheet showing a conventional, prior art process for the dehydration of ethanol.

FIG. 2 is a schematic flow sheet showing a process for the dehydration of ethanol according to the present invention.

FIG. 3 is a schematic flow sheet showing the temperature and pressure values for a simulated conventional process for the dehydration of ethanol.

FIG. 4 is a schematic flow sheet showing the temperature and pressure values for a simulated process for the dehydration of ethanol according to the present invention.

### DETAILED DESCRIPTION OF THE INVENTION

All parts, percentages and ratios used herein are expressed by volume unless otherwise specified. All documents cited herein are incorporated by reference.

By “water” it is meant any kind of water suitable for use in chemical and petrochemical processing, including deionized, demineralized, industrial, potable and distilled water.

By “steam” it is meant an inert gas, or a combination of non-condensable inert gases saturated with water vapor. “Superheated steam” is defined as a steam obtained by further heating a saturated steam at its saturation temperature.

Unless otherwise indicated, pressures are recited in values of MPa of absolute pressure.

The current invention introduces an ethanol dehydration process with improved energy efficiency. The reduced high-energy and steam consumption is obtained even using a high (“dilute”) steam to ethanol ratio to ensure a highly efficient process with nearly 100% conversion of the ethanol to ethylene. This ethanol dehydration process is especially suitable for producing ethylene oxide from ethanol.

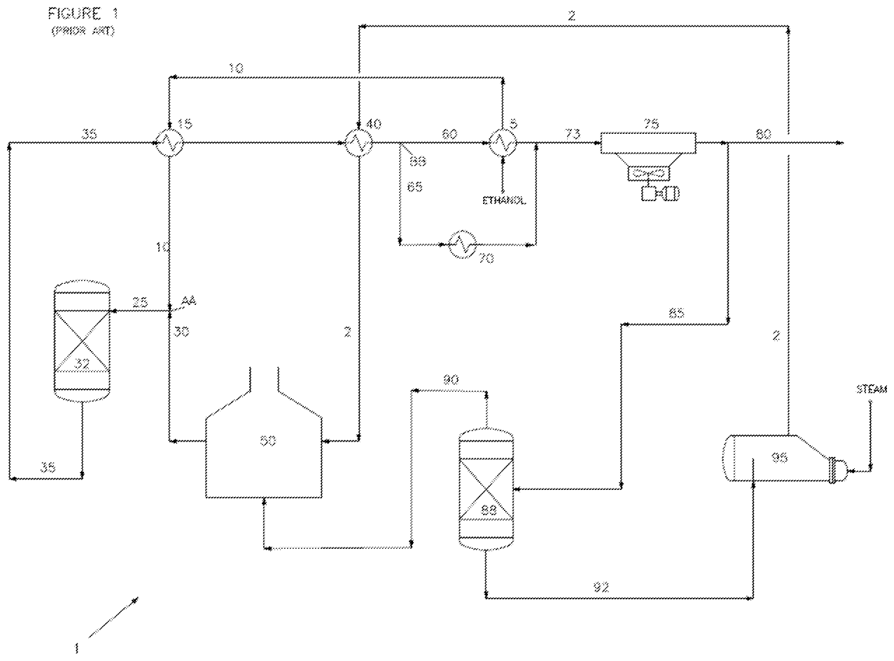

The process of the present invention will now be described with respect to FIG. 1, which shows a conventional, prior art ethanol dehydration process 1.

In process 1, a source of liquid ethanol is provided from an external source/OSBL at ambient temperature, preferably between about 10° C. to 30° C. The liquid ethanol passes through the exchanger 5 with the ethylene product stream 60 on the other side of the exchanger. The liquid ethanol is thus vaporized by indirect heat exchange with the ethylene product stream 60 in exchanger 5 resulting in an ethanol vapor stream 10. From heat exchanger 5 the ethanol vapor stream 10 passes through heat exchanger 15 with the ethylene product stream 35 on the other side of the exchanger 15. The ethanol vapor stream 10 is pre-heated in exchanger 15 to a temperature of between about 250 to 400° C., preferably between about 275 to 350° C. by indirect heat exchange with the ethylene product stream 35. A reactor feed mixture 25 is formed by blending the ethanol vapor stream 10 with a superheated steam stream 30 at point AA at a weight ratio of 1:1 to 1:5, preferably 1:2.5 to 1:3.5. (Higher ratios of steam dilution results in reduced conversion of ethanol in the dehydration reaction and should be avoided.) The resulting reactor feed mixture 25 has a temperature of about 425 to 500° C. and is fed or supplied at a pressure of about 0.15 to about 0.4 MPa (absolute) pressure to the ethanol dehydration reactor 32.

The ethanol dehydration reactor 32 is a single stage adiabatic reactor with a packed bed of dehydration catalyst. The reactor feed mixture (which, as described above, contains at least ethanol and steam) flows downward under pressure through the packed catalyst bed of the reactor 32 where the ethanol in the reactor feed mixture is dehydrated to ethylene at very high levels of selectivity and conversion %-resulting in a highly pure source of ethylene. The dehydration reaction is represented by the following equation:

C₂H₅OH→C₂H₄+H₂O+X

Where ‘X’ are by-products like aldehydes, ethers, ethanol, and C₃/C₄ compounds. Operating the process within optimal temperature and pressure ranges will limit the formation of these by-products while maximizing conversion and yield. Generally, conversion from ethanol to ethylene increases with the higher temperatures, and conversion falls—at times significantly—at higher pressures of the same. Thus, the aforementioned temperature and pressure ranges for the reactor feed mixture and dehydration reactor have been selected to maximize the selectivity and conversion of the dehydration reaction. It is important to note that ethanol itself can decompose into acetaldehyde at temperatures above 480° C. This acetaldehyde can cause damaging catalyst coking. Thus, if possible, the temperature of the reactor feed mixture should be maintained below about 480°.

As mentioned above, the reactor feed mixture 25, containing steam and ethanol, flows downward under pressure through the catalyst bed of the dehydration reactor 32 where ethanol is dehydrated to ethylene and produces an ethylene product stream 35 which is a gas and flows by pressure from the reactor. As mentioned above the dehydration reaction is operated at high rates of selectivity and conversion so that nearly all of the ethanol is converted to ethylene. Only trace amounts of unreacted ethanol and impurities like acetaldehyde are found in the ethylene product stream 35. So that in addition to ethylene, the ethylene product stream contains water plus trace amounts of unreacted ethanol and impurities like acetaldehyde. More specifically, the ethylene product stream 35 contains about 5 to about 20 mol % ethylene with the balance being water/steam and the unreacted ethanol and impurities. Measured on a non-aqueous basis, the ethylene product stream 35 contains about 95 mol % to about 99.5 mol % ethylene.

The ethylene product stream 35 leaves the reactor at a temperature of about 325 to 425° C. The ethylene product stream 35 is brought into thermal contact with the ethanol vapor stream 10 in exchanger 15 on the other side of the exchanger and thus, the ethanol vapor stream 10 is pre-heated by indirect heat exchange with the ethylene product stream 35, while at the same time the ethylene product stream 35 is cooled as a result of this thermal exchange. After leaving exchanger 15, the ethylene product stream 35 is brought into thermal contact with a process steam stream 2 at exchanger 40 which provides heat to pre-heat the process steam stream 2 to a temperature of about 275° C. to about 325° C. As a result of the thermal exchange in exchanger 40, the temperature of the ethylene product stream 35 is reduced to a range of between about 175 to 250° C.

The process steam stream 2 itself had originated from the steam generator 95, where it is created with the admixture of medium-pressure stream from OSBL. The process steam stream leaves the steam generator at a temperature of about 125 to 145° C. The process steam stream 2 is heated in heat exchanger 40, as described above then further heated in a furnace 50 to form a superheated steam stream 30. Stream 30 has a temperature of between about 500 to 650° C., preferably about 525 to 600° C. Stream 30 is then combined with steam 10 to form the reactor feed mixture 25 described above.

After exchanger 40, the ethylene-steam product stream is divided into two or more portions at point BB. FIG. 1 illustrates an embodiment in which the ethylene-steam vapor product stream is divided into exactly two portions at point BB: a first portion 60 is sent to exchanger 5 where it provides heat to vaporize the liquid ethanol stream 10. A second portion 65 is cooled further in exchanger 70 by providing export heat with a fluid (not shown) on the other side of the exchanger. This heated fluid can be used to enable downstream processes and preheating of utility steams (not shown).

By the time the second portion 65 of the ethylene-steam product stream has left exchanger 70, and has been combined with the first portion 60, the temperature of the recombined stream 73 has been significantly reduced: the temperature of the recombined stream is between about 90 to about 125° C. This cooling has been accomplished by the thermal transfer to exchanged streams in successive exchangers as previously described. At this point if there is no further heat requirement at downstream units, the recombined ethylene-steam product stream is sent to the dehydration condenser 75. In the dehydration condenser the recombined stream 73 is further cooled until condensed and thereby separated into a dehydration condensate 85 and a vapor ethylene product 80.

The ethylene in the vapor ethylene product 80 exceeds about 85 mol % ethylene, preferably exceeds about 90 mol %, with the balance being water preferably at saturation levels. This stream is then preferably compressed to a pressure range of about 0.5 to about 5 MPa (absolute) and optionally given a water wash and a caustic wash to remove remaining residual aldehydes, carbon dioxide, sulfur and other impurities before feeding it to downstream processes. For example, the ethylene product stream can be fed to an ethylene oxide reactor for the manufacture of ethylene oxide and derivatives of ethylene oxide.

The liquid water condensate 85 which contains some unreacted ethanol as well as other impurities such as acetaldehyde, is sent to a stripping column 88 where the ethanol, acetaldehyde and other impurities are stripped overhead into an impurities stream 90 and then sent to the ethanol dehydration furnace 50 for incineration (the impurities steam may be sent to a knock-out drum, not shown, prior to being sent to the furnace). This leaves a stripping column bottoms stream 92 comprising water and trace amounts of unreacted ethanol. The stripping column bottoms stream 92 is sent to the steam generator 95, where the process steam stream 2 required for the ethanol dehydration reactor is generated using medium-pressure steam supplied from OSBL to the steam generator 95. This process steam stream 2 is then sent for superheating in exchanger 40, as described above.

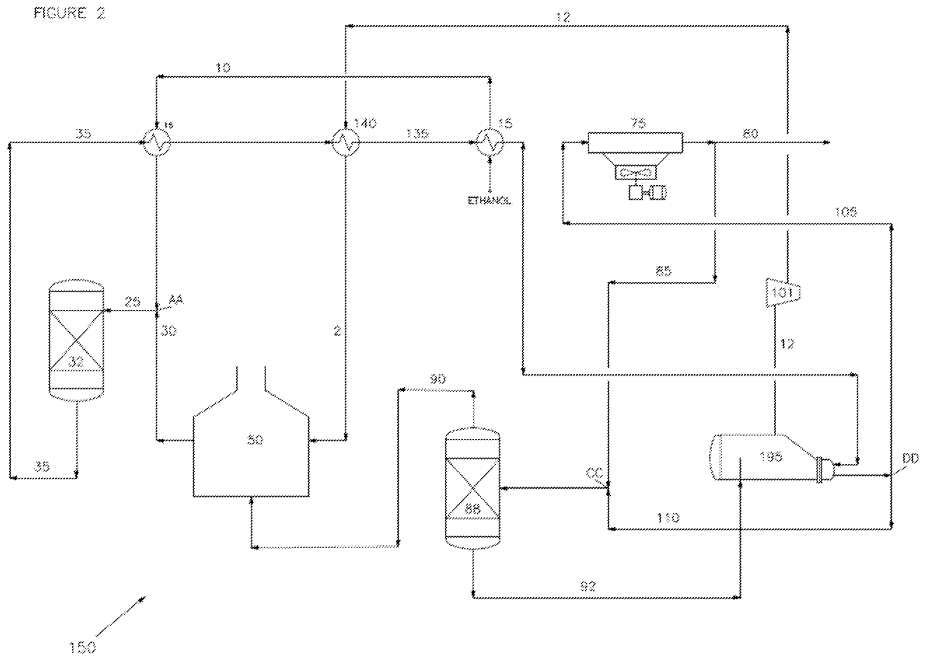

FIG. 2 shows a modified, inventive ethanol dehydration process 150 constructed according to the present invention. While the conventional process 1 shown in FIG. 1 combines both excellent heat integration with ethanol dehydration at high rates of selectivity and conversion, process 1 requires a continual import of steam that increases the utility cost of the process. Inventive process 150 differs from the conventional process in a number of ways but especially in that by making use of the latent heat released from the condensation of steam, process 150 eliminates the need for continual medium-pressure steam import during operation to generate the process steam used in the process. (It should be noted while continual steam import is not necessary during operation, it nonetheless may be necessary at other times, such as during start-up and may occasionally be selected by the plant operator under certain circumstances or during occasional operational excursions.)

Further to the brief description of the inventive process 150, above, a more detailed description of the inventive process 150 will now be made with reference to the conventional process 1. Many of the same streams, services, and equipment items that are present in process 1 are also present in process 150 within the same temperature ranges, pressure ranges, stream composition and other ranges, thus the units and streams in process 150 have the same values as those identically numbered and shown in 1, unless otherwise indicated.

In the inventive process, the ethanol product stream 35 is maintained within the same temperature and pressure ranges as disclosed previously with respect to the inventive process. However, a difference can be seen in the present invention when the ethanol product stream 35 enters the heat exchanger 140. Working backwards in the present invention, the second process steam stream 12 having just been compressed in the dehydration steam compressor 101 comes into the heat exchanger 40 much warmer than the corresponding stream 2 in the conventional process. As a consequence, the ethanol product stream 35 as it is passed through heat exchanger 140 is increased to even higher temperatures. As shown in FIG. 2, the stream 35 passes through the heat exchanger 140 where it is brought into thermal contact with the second process steam stream 12 on the other side of the exchanger 140 and passes out of the exchanger as the high temperature ethanol vapor stream 135, having a temperature of between about 250° C. to about 325° C. (The formation and temperature and pressure of the second process steam stream 12 is described in greater detail, below.)

The high temperature ethanol vapor stream 135 passes from heat exchanger 140 and flows through the one side of the heat exchanger 15 where it exchanges heat with the liquid ethanol on the other side of the exchanger and vaporizes the ethanol to produce the ethanol vapor stream 10 as described with respect to the process shown in FIG. 1. Here, the present invention makes a major change in operation compared to conventional processes in that after leaving exchanger 15, 100% of the stream flows to the second steam generator exchanger 195, rather than some being diverted for supply of heat and utility supply (by contrast, see the diversion at point BB in FIG. 1 and the accompanying description). This change in operation in the present invention can be seen in FIG. 2 where after thermal exchange with the liquid ethanol stream the temperature of stream 135 is now reduced to the range of about 125 to 140° C.; and then the entire high temperature ethanol vapor stream 135 is sent to the second steam generator 195. It flows at a pressure of about 0.2 to about 0.3 MPa (absolute) to the tube side of the steam generator heat exchanger 195, where it cools sufficiently within 195 to reach the dew point for the product ethylene stream of approximately 120° C. As a result, the vapor in the product ethylene stream condenses, releasing the heat of condensation, which is harvested for generating steam to form the second process steam stream 12. The result of this is considerable energy savings, as it is no longer necessary to import steam to provide heat for the process. This savings greatly exceeds any loss of heat integration from the product ethylene stream no longer being available to provide heat to downstream sources. This is because the amount of heat available from the latent heat of condensation to generate steam in the steam generator 195 greatly exceeds the amount of heat in process 1 that can be thermally transferred as sensible heat in further exchangers.

The ethylene product stream then passes out from the steam generator as a result of the pressure gradient across the steam generator and ethylene product stream is separated by flashing into a steam generator vapor stream 105 and a steam generator condensate 110 at point DD. The steam generator condensate flows from the steam generator 195 and is blended with the liquid water condensate at CC and then the combined stream is sent to the stripping column 88, which is operated as described above for process 1. The remaining steam generator vapor stream 105 flows to the dehydration condenser 75, which is operated as described above with respect to process 1.

The second process steam stream 12 of process 150 leaves the steam generator at a temperature of about 95° C. to about 120° C. comparable to the temperature of process steam stream 12 in process 1, but unlike in process 1, no import of medium-pressure steam was necessary. However, without the addition of the medium pressure steam, the pressure is lower on the shell side steam generator and the process steam stream 2 is pressurized only to a pressure of about 0.125 MPa to about 0.20 MPa (absolute) as it leaves the steam generator. Accordingly in order to make the process steam stream useful in the rest of process 150, the process steam stream is compressed by a dehydration steam compressor 101 to a pressure of about 0.3 MPa to about 0.45 MPa (absolute). After passing through the compressor the temperature is correspondingly raised to between about 225° C. to about 275° C.

As mentioned above, an important input to processes 1 and 150 is ethanol, which is supplied from an external source/OSBL. It is especially preferred that the ethanol is bioethanol, meaning it is produced from biomass material. Bioethanol itself is obtained by fermentation of plant material: vegetable biomass and agricultural byproducts and wastes; all of which are abundant and renewable. The fermentation of biomass to ethanol results in mixtures containing about 95% water and 5% ethanol. The water may then be separated out using a combination of azeotropic distillation or solvent extraction.

The dehydration reactor of the presentation invention may make use of any suitable dehydration catalyst including heterogeneous catalysts such as zeolites, γ-alumina (Al₂O₃) and the like. Particularly preferred is the γ-alumina dehydration catalyst sold under the trade name SynDol by Scientific Design Company, Inc.

As mentioned above, the vaporous ethylene in stream 80 may be further refined and then used downstream to produce ethylene oxide. Ethylene oxide is produced by continuously contacting an oxygen-containing gas with the ethylene produced as described above in the presence of a silver-based ethylene oxide (“epoxidation”) catalyst), in a fixed-bed tubular reactor. (The silver-based epoxidation catalyst described in greater detail below.) Oxygen may be supplied to the reaction in substantially pure molecular form or in a mixture such as air. By way of example, typical reactant feed mixtures under operating conditions may contain from about 0.5% to about 45%, preferably about 5% to about 30% of ethylene and from about 3% to about 15% oxygen, and from about 0.3% to about 10% carbon dioxide with the balance comprising comparatively inert materials, including such substances as water, inert gases, other hydrocarbons, and the reaction moderators described herein. Non-limiting examples of inert gases include nitrogen, argon, helium and mixtures thereof. Non-limiting examples of the other hydrocarbons include methane, ethane, propane and mixtures thereof. Carbon dioxide and water are byproducts of the epoxidation process as well as common contaminants in the feed gases. Both have adverse effects on the catalyst, so the concentrations of these components are usually kept at a minimum.

Also present in the reaction, as previously mentioned, are one or more reaction moderators, non-limiting examples of which include organic halogen-containing compounds such as C1 to C8 halohydrocarbons; especially preferred are chloride-containing moderators such as methyl chloride, ethyl chloride, ethylene dichloride, vinyl chloride or mixtures thereof. Controlling chloride concentration level is particularly important with rhenium-containing catalysts.

As mentioned above, a usual method for the ethylene epoxidation process comprises the contacting ethylene with oxygen, in the presence of an epoxidation catalyst, in a fixed-bed tubular reactor. Conventional, commercial fixed-bed ethylene-oxide reactors are typically in the form of a plurality of parallel elongated tubes (in a suitable shell) approximately 0.7 to 2.7 inches O.D. and 0.5 to 2.5 inches I.D. and 15-53 feet long, each filled and packed with catalyst. The reaction feed mixture (described above) is introduced into these tubes, and the resulting reactor effluent gas contains ethylene oxide, un-used reactants, and byproducts. Typically in the ethylene oxide process, the work rate for the reactor is between 130 and 300 kg/m³/h, while the ΔEO is between 1.0% and 2.5%. The work rate is the production rate and is represented herein by the units kg/m³/h. The ΔEO is defined as the moles of EO formed in the reactor per 100 moles of reactor feed and essentially represents the concentration of ethylene oxide in the reactor effluent, since the concentration of ethylene oxide in reactor feed must be maintained at very close to zero, indeed typically only a few ppm. The feed composition in the reactor inlet after the completion of start-up and during normal operation typically comprises (by volume %) 1-40% ethylene, 3-12% O2; 0.3% to 20%, preferably 0.3 to 5%, more preferably 0.3 to 1% of CO2; 0-3% ethane, an amount of one or more chloride moderators, which are described herein; and the balance of the feed being comprised of argon, methane, nitrogen or mixtures thereof.

Typical operating temperatures for the ethylene epoxidation process involve temperatures in the range from about 180° C. to about 330° C., and preferably, from about 200° C. to about 325° C., and more preferably from about 225° C. to about 280° C. The operating pressure may vary from about atmospheric pressure to about 30 atmospheres, depending on the mass velocity and productivity desired. Higher pressures may be employed within the scope of the invention. Residence times in commercial-scale reactors are generally on the order of about 2 to about 20 seconds.

### Silver-Based Epoxidation Catalyst

As mentioned above, the present ethylene oxide process makes use of a silver-based epoxidation catalyst. The silver-based epoxidation catalyst includes a support, and at least a catalytically effective amount of silver or a silver-containing compound; also optionally present is a promoting amount of rhenium or a rhenium-containing compound; also optionally present is a promoting amount of one or more alkali metals or alkali-metal-containing compounds. The support employed in this invention may be selected from a large number of solid, refractory supports that may be porous and may provide the preferred pore structure. Alumina is well known to be useful as a catalyst support for the epoxidation of an olefin and is the preferred support.

Regardless of the character of the support used, it is usually shaped into particles, chunks, pieces, pellets, rings, spheres, wagon wheels, cross-partitioned hollow cylinders, and the like, of a size suitable for employment in a fixed-bed epoxidation reactor. The support particles will preferably have equivalent diameters in the range from about 3 mm to about 12 mm, and more preferably in the range from about 5 mm to about 10 mm. (Equivalent diameter is the diameter of a sphere having the same external surface (i.e., neglecting surface within the pores of the particle) to volume ratio as the support particles being employed.) Suitable supports are available from Saint-Gobain Norpro Co., Sud Chemie AG, Noritake Co., CeramTec AG, and Industrie Bitossi S.p.A. Without being limited to the specific compositions and formulations contained therein, further information on support compositions and methods for making supports may be found in U.S. Patent Publication No. 2007/0037991.

In order to produce a catalyst for the oxidation of an olefin to an olefin oxide, a support having the above characteristics is then provided with a catalytically effective amount of silver on its surface. In one embodiment, the catalytic effective amount of silver is from 10% by weight to 45% by weight. The catalyst is prepared by impregnating the support with a silver compound, complex or salt dissolved in a suitable solvent sufficient to cause deposition of a silver-precursor compound onto the support. Preferably, an aqueous silver solution is used.

A promoting amount of a rhenium component, which may be a rhenium-containing compound or a rhenium-containing complex may also be deposited on the support, either prior to, coincidentally with, or subsequent to the deposition of the silver. The rhenium promoter may be present in an amount from about 0.001 wt. % to about 1 wt. %, preferably from about 0.005 wt. % to about 0.5 wt. %, and more preferably from about 0.01 wt. % to about 0.1 wt. % based on the weight of the total catalyst including the support, expressed as the rhenium metal.

Other components which may also be deposited on the support either prior to, coincidentally with, or subsequent to the deposition of the silver and rhenium are promoting amounts of an alkali metal or mixtures of two or more alkali metals, as well as optional promoting amounts of a Group IIA alkaline earth metal component or mixtures of two or more Group IIA alkaline earth metal components, and/or a transition metal component or mixtures of two or more transition metal components, all of which may be in the form of metal ions, metal compounds, metal complexes and/or metal salts dissolved in an appropriate solvent. The support may be impregnated at the same time or in separate steps with the various catalyst promoters. The particular combination of support, silver, alkali metal promoter(s), rhenium component, and optional additional promoter(s) of the instant invention will provide an improvement in one or more catalytic properties over the same combination of silver and support and none, or only one of the promoters.

As used herein the term “promoting amount” of a certain component of the catalyst refers to an amount of that component that works effectively to improve the catalytic performance of the catalyst when compared to a catalyst that does not contain that component. The exact concentrations employed, of course, will depend on, among other factors, the desired silver content, the nature of the support, the viscosity of the liquid, and solubility of the particular compound used to deliver the promoter into the impregnating solution. Examples of catalytic properties include, inter alia, operability (resistance to runaway), selectivity, activity, conversion, stability and yield. It is understood by one skilled in the art that one or more of the individual catalytic properties may be enhanced by the “promoting amount” while other catalytic properties may or may not be enhanced or may even be diminished.

Suitable alkali metal promoters may be selected from lithium, sodium, potassium, rubidium, cesium or combinations thereof, with cesium being preferred, and combinations of cesium with other alkali metals being especially preferred. The amount of alkali metal deposited or present on the support is to be a promoting amount. Preferably, the amount ranges from about 10 ppm to about 3000 ppm, more preferably from about 15 ppm to about 2000 ppm, and even more preferably from about 20 ppm to about 1500 ppm, and as especially preferred from about 50 ppm to about 1000 ppm by weight of the total catalyst, measured as the metal.

Suitable alkaline earth metal promoters comprise elements from Group IIA of the Periodic Table of the Elements, which may be beryllium, magnesium, calcium, strontium, and barium or combinations thereof. Suitable transition metal promoters may comprise elements from Groups IVA, VA, VIA, VIIA and VIIIA of the Periodic Table of the Elements, and combinations thereof.

The amount of alkaline earth metal promoter(s) and/or transition metal promoter(s) deposited on the support is a promoting amount. The transition metal promoter may typically be present in an amount from about 0.1 micromoles per gram to about 10 micromoles per gram, preferably from about 0.2 micromoles per gram to about 5 micromoles per gram.

The silver solution used to impregnate the support may also comprise an optional solvent or a complexing/solubilizing agent such as are known in the art. A wide variety of solvents or complexing/solubilizing agents may be employed to solubilize silver to the desired concentration in the impregnating medium. Useful complexing/solubilizing agents include amines, ammonia, oxalic acid, lactic acid and combinations thereof. Amines include an alkylene diamine having from 1 to 5 carbon atoms. In one preferred embodiment, the solution comprises an aqueous solution of silver oxalate and ethylene diamine. The complexing/solubilizing agent may be present in the impregnating solution in an amount from about 0.1 to about 5.0 moles per mole of silver, preferably from about 0.2 to about 4.0 moles, and more preferably from about 0.3 to about 3.0 moles for each mole of silver.

When a solvent is used, it may be an organic solvent or water, and may be polar or substantially or totally non-polar. In general, the solvent should have sufficient solvating power to solubilize the solution components. At the same time, it is preferred that the solvent be chosen to avoid having an undue influence on or interaction with the solvated promoters. Organic-based solvents which have 1 to about 8 carbon atoms per molecule are preferred. Mixtures of several organic solvents or mixtures of organic solvent(s) with water may be used, provided that such mixed solvents function as desired herein.

The concentration of silver in the impregnating solution is typically in the range from about 0.1% by weight up to the maximum solubility afforded by the particular solvent/solubilizing agent combination employed. It is generally very suitable to employ solutions containing from 0.5% to about 45% by weight of silver, with concentrations from 5 to 35% by weight of silver being preferred.

Impregnation of the selected support is achieved using any of the conventional methods; for example, excess solution impregnation, incipient wetness impregnation, spray coating, etc. Typically, the support material is placed in contact with the silver-containing solution until a sufficient amount of the solution is absorbed by the support. Preferably the quantity of the silver-containing solution used to impregnate the porous support is no more than is necessary to fill the pores of the support. A single impregnation or a series of impregnations, with or without intermediate drying, may be used, depending, in part, on the concentration of the silver component in the solution. Impregnation procedures are described, for example, in U.S. Pat. Nos. 4,761,394, 4,766,105, 4,908,343, 5,057,481, 5,187,140, 5,102,848, 5,011,807, 5,099,041 and 5,407,888. Known prior procedures of pre-deposition, co-deposition and post-deposition of various the promoters can be employed.

After impregnation of the support with the silver-containing compound, i.e., a silver precursor, a rhenium component, an alkali metal component, and the optional other promoters, the impregnated support is calcined for a time sufficient to convert the silver containing compound to an active silver species and to remove the volatile components from the impregnated support to result in a catalyst precursor. The calcination may be accomplished by heating the impregnated support, preferably at a gradual rate, to a temperature in the range from about 200° C. to about 600° C. at a pressure in the range from about 0.5 to about 35 bar. In general, the higher the temperature, the shorter the required heating period. A wide range of heating periods have been suggested in the art; e.g., U.S. Pat. No. 3,563,914 discloses heating for less than 300 seconds, and U.S. Pat. No. 3,702,259 discloses heating from 2 to 8 hours at a temperature of from 100° C. to 375° C., usually for duration of from about 0.5 to about 8 hours. However, it is only important that the heating time be correlated with the temperature such that substantially all of the contained silver is converted to the active silver species. Continuous or step-wise heating may be used for this purpose.

During calcination, the impregnated support may be exposed to a gas atmosphere comprising an inert gas or a mixture of an inert gas with from about 10 ppm to 21% by volume of an oxygen-containing oxidizing component. For purposes of this invention, an inert gas is defined as a gas that does not substantially react with the catalyst or catalyst precursor under the conditions chosen for the calcination. Further information on catalyst manufacture may be found in the aforementioned U.S. Patent Publication No. 2007/0037991.

### Examples

The invention will now be described in more detail with respect to the following non-limiting simulated examples.

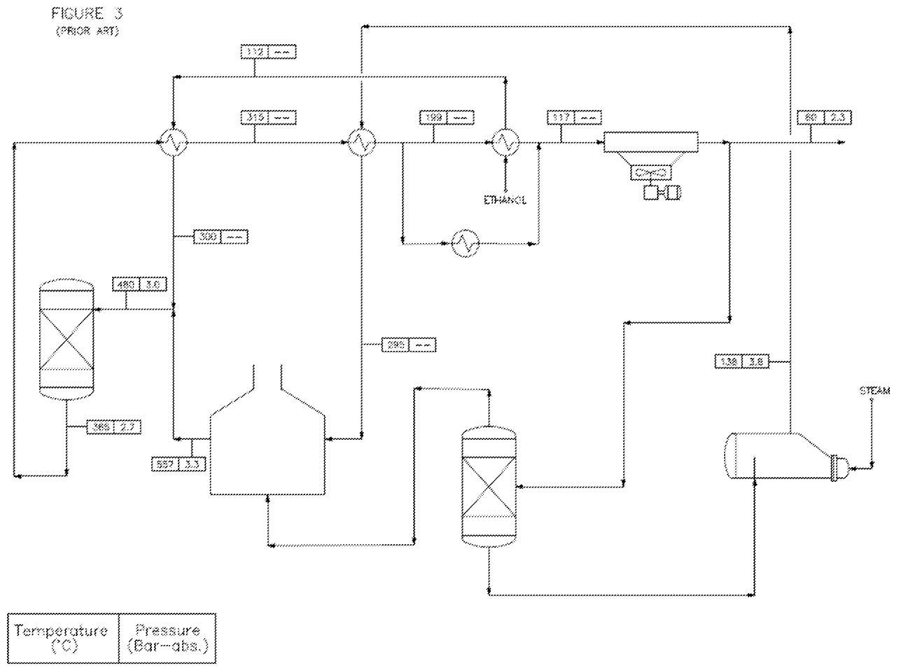

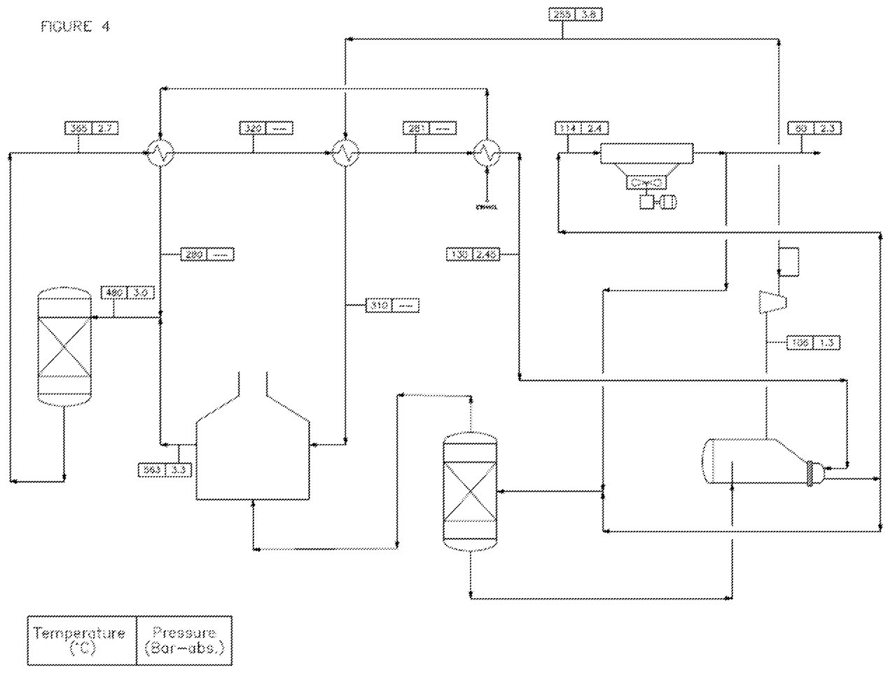

Ethanol dehydration processes prepared according to a conventional ethanol dehydration process 1 and according to the present invention 150 are shown in FIGS. 1 and 2, respectively, and are simulated using PRO/II software. The temperatures and pressure of each stream used in the simulations are shown in FIG. 3 (simulation of process 1, illustrated in FIG. 1) and FIG. 4 (simulation of process 150, illustrated in FIG. 2).

In addition to the temperature and pressure values indicated in FIGS. 3-4, the following reactor parameters are also part of the simulation:

TABLE I

| | Conventional | Inventive |
|---|---|---|
| | Process (#1) | Process (#150) |
| Temp., ° C. (inlet) | 480 | 480 |
| WHSV, h⁻¹ | 0.30 | 0.30 |
| Ethanol Conversion | 99.8% | 99.8% |
| Ethylene Selectivity | 99.4% | 99.4% |

Attention is drawn particularly to a comparison of the temperature and pressure values for Process Steam Stream 2 (in process 1) and Second Process Steam Stream 12 (in process 150). As mentioned above, in the conventional ethanol dehydration process 1 none of the ethylene-steam product stream is directed to the steam generator, but rather all of it sent to the dehydration condenser and then immediately leaves the process for further purification and/or use in downstream processes. Accordingly, in the conventional process the import of steam is necessary to provide heat and steam for the process. As a result of the steam import, the temperature and pressure of the process steam stream 2 in the conventional process 1 are higher than the corresponding stream 12 in the process according the present invention 150, which does not import steam, this is shown in Table II:

TABLE II

| | Temperature (° C.) | Pressure (MPa) |
|---|---|---|
| Process steam stream 2 | 138 | 3.80 |
| (Conventional Process #1) | | |
| Second process steam stream 12 | 106 | 1.30 |
| (Inventive Process #150) | | |

The benefits of the present invention can be seen particularly in Table III, which compares the energy balance for the simulated conventional ethanol dehydration process 1 with that of the simulated present invention 150. The dehydration process of the present invention has an energy requirement of 14.45 MKal/hr, which is far lower than the 41.84 of the simulated conventional process. This savings is primarily because imported steam is no longer required. The exact differences in energy requirements are noted in Table III, below.

TABLE III

| | | Conventional | Inventive |
|---|---|---|---|
| | Unit | Process (#1) | Process (#150) |
| Heat In | | | |
| Ethanol dehydration furnace | Mkcal/hr | 8.04 | 8.05 |
| Ethanol vaporizer | Mkcal/hr | | |
| Dehydration steam | Mkcal/hr | | 4.5 |
| compressor | | | |
| Imported Steam to Steam | Mkcal/hr | 32.6 | |
| generator | | | |
| Aldehyde stripper reboiler | Mkcal/hr | 1.2 | 2.9 |
| Total | | 41.84 | 14.45 |

It should be noted that in addition to the huge savings seen in the above table, there is also an additional energy requirement to power the steam compressor. However, this energy requirement, 4.5 Mkal/hr, is small compared to the energy cost of the imported steam. Additionally, it should be noted that in the inventive process there is no heat available for export, as shown in Table IV, below.

TABLE IV

| | | Conventional | Inventive |
|---|---|---|---|
| Heat Out | Unit | Process (#1) | Process (#150) |
| Endothermic Heat of | Mkcal/hr | 5.5 | 5.4 |
| reaction | | | |
| Waste heat boiler | Mkcal/hr | | |
| Quench tower | Mkcal/hr | | |
| Export heat | Mkcal/hr | 28.2 | |
| Dehydration condenser | Mkcal/hr | 8.14 | 9.05 |
| (air cooler) | | | |
| Total | | 41.84 | 14.45 |

However, this export heat has little value for use in downstream processes and so its loss does not diminish the considerably improved process economics of the inventive process.

We claim:

1. A process for the dehydration of ethanol to ethylene comprising the steps of:
(a) forming a reactor feed mixture by blending an ethanol vapor stream with a superheated steam stream;
(b) supplying the reactor feed mixture to the ethanol dehydration reactor;
(c) dehydrating the ethanol in the reactor feed mixture in ethanol dehydration reactor to form an ethylene product stream;
(d) supplying the ethylene product stream to a steam generator at a pressure of about 0.2 to about 0.3 MPa and a temperature of about 125° C. to about 140° C.;
(e) cooling the ethylene product stream to below a dew point of the ethylene product stream to release the heat of condensation;
(f) generating a second process steam stream by the heat of condensation, the process steam stream having a temperature of about 95° C. to about 120° C.;
(g) passing from the steam generator the second process steam stream at a pressure of about 0.125 MPa to about 0.2 MPa; and
(h) compressing the second process steam stream to a pressure of about 0.3 MPa to about 0.45 MPa.

2. The process of claim 1 further comprising the steps of:
directing the ethylene product stream outward from the steam generator; and
separating by flashing the ethylene product stream into a steam generator condensate and a steam generator vapor stream.

3. The process of claim 1 wherein the ethanol dehydration reactor is a single stage adiabatic reactor with a packed bed of dehydration catalyst.

4. The process of claim 1 wherein the ethanol dehydration reactor is a single stage adiabatic reactor with a packed bed of γ-alumina dehydration catalyst.

5. The process of claim 1 wherein the reactor feed mixture has a temperature of about 425 to about 500° C. and the ethylene product stream leaves the reactor at a temperature of about 325 to 425° C.

6. The process of claim 1, wherein in step (a) the reactor feed mixture is formed by blending the ethanol vapor stream with a superheated steam stream at a weight ratio of 1:1 to 1:5.

7. The process of claim 1, wherein the ethylene product stream contains about 95 mol % to about 99.5 mol % ethylene on a non-aqueous basis.

8. The process of claim 1, wherein the process further comprises preheating the ethanol vapor stream in a heat-exchanger by thermal exchange with the ethylene product stream.

9. The process of claim 1, wherein after the compressing step the temperature of the process steam stream is about 225° C. to about 275° C.

10. The process of claim 1, wherein the ethylene product stream has a temperature of about 325 to 425° C.

11. The process of claim 1, wherein the ethylene product stream contains from about 95 mol % to about 99.5 mol % ethylene, on a non-aqueous basis.

12. The process of claim 1, wherein the temperature of the reactor feed mixture is below about 480°.

13. The process of claim 1, wherein the reactor feed mixture is supplied at a pressure of about 0.15 to about 0.4 MPa (absolute) to the ethanol dehydration reactor.

14. The process of claim 1, wherein the ethylene is subjected to a caustic wash to remove impurities.

15. The process of claim 1 where bioethanol is the source of the ethanol vapor stream.

16. The process of claim 1, wherein the ethylene is contacted with oxygen in the presence of a silver-based epoxidation catalyst in a fixed-bed tubular reactor to produce ethylene oxide.

17. The process of claim 1, wherein the ethylene is contacted with oxygen in the presence of a silver-based epoxidation catalyst in a fixed-bed tubular reactor to produce ethylene oxide, wherein the reactor is operated at a work rate of between about 130 and about 300 kg/m³/h.

18. The process of claim 1, wherein the silver-based epoxidation catalyst contains about 15 wt % to about 40 wt % silver.

19. The process of claim 1, wherein the ethylene is contacted with oxygen in the presence of a silver-based epoxidation catalyst in a fixed-bed tubular reactor to produce ethylene oxide, wherein the reactor is operated at a temperature of between about 240° C. to about 280° C.

20. A process for the dehydration of ethanol to ethylene comprising the steps of:
(a) forming a reactor feed mixture by blending an ethanol vapor stream with a superheated steam stream at a weight ratio of 1:1 to 1:5, the reactor feed mixture having a temperature of about 425 to about 500° C.;
(b) supplying the reactor feed mixture to the ethanol dehydration reactor, wherein the ethanol dehydration reactor is a single stage adiabatic reactor with a packed bed of γ-alumina dehydration catalyst;
(c) dehydrating the ethanol in the reactor feed mixture in ethanol dehydration reactor to form an ethylene product stream, the ethylene product stream having a temperature of about 325° C. to about 425° C. upon leaving the reactor;
(d) supplying the ethylene product stream to a steam generator at a pressure of about 0.2 to about 0.3 MPa and a temperature of about 125° C. to about 140° C.;
(e) cooling the ethylene product stream to below a dew point of the ethylene product stream to release the heat of condensation;
(f) generating a second process steam stream by the heat of condensation, the process steam stream having a temperature of about 95° C. to about 120° C.;
(g) passing from the steam generator the second process steam stream at a pressure of about 0.125 MPa to about 0.20 MPa; and
(h) compressing the second process steam stream to a pressure of about 0.3 MPa to about 0.45 MPa.
